(19) United States
(12) Patent Application Publication
(10) Pub. No.: US 2005/0079534 A1
(43) Pub. Date: Apr. 14, 2005

(54) Lysis and neutralization method for production of pharmaceutical grade plasmid DNA

(75) Inventors: R. Monty Warthen, Salisbury, MD (US); Nallakannu Lakshmanan, Millsboro, DE (US)

Correspondence Address: AKZO NOBEL PHARMA PATENT DEPARTMENT, PO BOX 318, MILLSBORO, DE, 19966, US

(73) Assignee: AKZO NOBEL, N.V.

(21) Appl. No.: 10/961,938

(22) Filed: Oct. 8, 2004

Related U.S. Application Data
(60) Provisional application No. 60/509,718, filed on Oct. 8, 2003.

(51) Int. Cl. C12Q 1/68; C12N 15/74

(52) U.S. Cl. 435/6; 435/320.1

(57) ABSTRACT

The present invention is a short duration lysis and neutralization process developed for the production of pharmaceutical grade plasmid DNA. This process includes the mixing of concentrated bacterial suspension and lysis solution through a “T” or “Y” shaped device, followed by the addition of neutralization solution through a another “T” or “Y” shaped device. A thorough mixing of bacterial suspension, lysis solution, and neutralization solution was achieved by addition of static mixer(s) at post-lysis and post-neutralization steps. The neutralized bacterial suspension was clarified by bag-filtration to remove macromolecules and host cell chromosomal DNA-protein precipitates.

### FIELD OF INVENTION

The present invention is a short time duration lysis and neutralization process developed for the production of pharmaceutical grade plasmid DNA. This process includes the mixing of concentrated bacterial suspension and lysis solution through a “T” or “Y” shaped device, followed by the addition of neutralization solution through a another “T” or “Y” shaped device. A thorough mixing of bacterial suspension, lysis solution, and neutralization solution was achieved by addition of static mixers at post-lysis and post-neutralization steps. The neutralized bacterial suspension was clarified by bag-filtration to remove macromolecules and host cell chromosomal DNA-protein precipitates. Contaminants such as fragmented host cell chromosomal DNA, RNA, endotoxin, etc. were removed by a combination of TFF and chromatography methods. There were no centrifugation or precipitation steps used in this process. The lysis and neutralization process has a short time duration and is easily scaleable to multi-liter fermentation cultures.

### BACKGROUND OF THE INVENTION

It has been shown that plasmid DNA can be used as a non-viral gene delivery system for clinical applications (Wang et. al 1993). Plasmid-based genes offer promise for a new generation of vaccines and for gene therapy. For gene therapy and genetic immunization, the plasmid DNA themselves rather than the expressed proteins are the desired pharmaceutical products. Therefore, a need for the development of large scale plasmid DNA production and purification processes for the production of pharmaceutical grade plasmid DNA does exist.

A number of fermentation conditions and fermentation techniques are available for large-scale production of E. coli (Fieschko et al., 1986; Riesenberg et al., 1991; Wei, 1999). Several methods are available for isolation and purification of plasmid from host cells (Birnboim and Doly, 1979; Maniatis et al 1982; Bussey et al., 2000; Theodossiou and Dunnill, 1999; Morsey, 1999; Thatcher et al., 1999, Ferreira et al., 2000).

The most commonly used method for isolation of plasmid DNA from the host cell is the method described by Birmboim and Doly (1979). The principle of the method is selective alkaline denaturation of high molecular weight chromosomal DNA while covalently closed circular DNA remains double-stranded. Upon neutralization, the chromosomal DNA renatures to form an insoluble clot, leaving the plasmid DNA in aqueous supernatant. The insoluble precipiate is separated from the aqueous phase either by filtration or by centrifugation. The plasmid DNA in the aqueous solution is isolated by alcohol precipitation and purified either by centrifugation using cesium chloride (Maniatis et al., 1982) or by an anion exchange chromatography.

Lee and Sager proposed a high temperature method to denature and precipitate the host cell proteins and chromosomal DNA in the cell lysate (Lee and Sager, 2001). In this technique the cell slurry resulting from the addition of bacterial cells and lysis solution was heated to 65° C. to 93° C. in a heat exchanger to denature cell proteins. Centrifugation or filtration was used to remove the cell debris and clots. RNAse was used to degrade the host cell RNA in the aqueous phase. The plasmid DNA was purified with an anion exchange matrix.

Host cell RNA is one of the major contaminants in the cell lysate and it represents approximately 20% of the total dry weight of E. coli (Neidhardt and Umbarger, 1996). RNAse treatment is a commonly used method to degrade bacterial RNA in the cell lysate. The degraded RNA in the aqueous phase is then removed during the purification of plasmid DNA either by certification or by chromatography. Both recombinant and non-recombinant RNAses are available for removal of host cell RNA in the plasmid purification. The most commonly used form of RNAse is of bovine origin.

There is an increasing concern regarding the use of bovine RNAse due to the prevalence of spongiform encephalopathies (Hill et la., 1997). RNAse-free plasmid DNA isolation methods were described by Butler et al. (U.S. Pat. No. 6,313,285, 2001) and Ferreira et al. (1999). Lysis time longer than 4 hours to degrade the contaminating RNA was proposed by Butler et al. The degraded RNA was then removed by tangential flow filtration (TFF). Sequential clarification with chaotropic salts, concentration with PEG, and ion-exchange and size-exclusion chromatography was proposed as an alternative method for removal RNA by Ferreira et al. (1999).

Higher lysis pH and/or longer incubation time in lysis solution is the probable cause for the formation of irreversibly denatured (“trefoil”) plasmid DNA. The irreversibly denatured form of plasmid has high mobility on gel electrophoresis compared to reversibly denatured form of plasmid of same size and is also one of the causes of poor recovery of plasmid during the plasmid isolation and purification (Thatcher et al., 1997). The irreversibly denatured plasmid DNA is biologically non-functional. The shorter incubation time in the present invention helps prevent or reduce “trefoil” formation.

Tangential flow filtration (TFF), or cross-flow filtration, is a separation technique whereby flow is directed across the membrane surface in a sweeping motion (Gabler, ASM News, 50: 299 (1984)). This sweeping action helps to keep material retained by the membrane from creating a layer on the filter surface, a condition known as concentration polarization. TFF is used to concentrate and/or desalt solutions retained by the membrane (retentate) or to collect material passing through the membrane (filtrate). Materials smaller than the pore size (or nominal-molecular-weight cutoff (NMWC)) are able to pass through the membrane and may be depyrogenated, clarified, or separated from higher-molecular-weight or larger species. Materials larger than the pore size or NMWC are retained by the membrane and are concentrated, washed, or separated from the low-molecular-weight species. The principles, theory, and devices used for TFF are described in Michaels et al., “Tangential Flow Filtration” in Separations Technology, Pharmaceutical and Biotechnology Applications (W. P. Olson, ed., Interpharm Press, Inc., Buffalo Grove, Ill. 1995).

There are many challenges associated with downstream processing for purification of pharmaceutical grade plasmid DNA. Many laboratory scale methods for purification of plasmid DNA utilize techniques and chemicals that are unsuitable for development of large scale production.

### BRIEF DESCRIPTION OF THE FIGURES

FIG. 1. The influence of lysis time on plasmid quality and quantity.

FIG. 2. The influence of neutralization time on plasmid quality and quantity.

FIG. 3. The effect of NaOH concentration lysis buffer volume and lysis time on plasmid quality and plasmid yield.

FIG. 4. “In-line” lysis and neutralization assembly.

FIG. 5. “In-Line” lysis and neutralization: The effect of NaOH concentration and lysis buffer volume on plasmid quality and plasmid yield.

FIG. 6. The location of motionless mixture.

FIG. 7. The effect of addition of motionless mixture on plasmid quality and plasmid yield.

FIG. 8. The location of motionless mixture.

FIG. 9. The effect of addition of motionless mixture on plasmid quality and plasmid yield.

FIG. 10. A lab-scale version of in-line lysis and neutralization assembly with motionless mixtures

### DETAILED DESCRIPTION OF THE INVENTION

A novel continuous short duration lysis and neutralization in combination with TFF and column chromatography protocol is described here which utilizes RNAse free processes for the production of pharmaceutical grade plasmid DNA. The quality of the plasmid produced by this process is comparable or superior to standard protocols used for the purification and isolation of plasmid DNA. Another embodiment of the present invention is a batch process.

Definitions:

“Filtrate” refers to that portion of a sample that passes through the filtration membrane.

“Retentate” refers to that portion of a sample that does not pass through the filtration membrane.

“Tangential flow filtration” or “TFF” or “crossflow filtration” refers to a filtration process in which the sample mixture circulates across the top of the membrane, while applied pressure causes solute and small molecules to pass through the membrane.

“Lysate contaminants” refers to all undesired components of a mixture in which the desired plasmid DNA is contained, including chromosomal DNA, host proteins, cell debris, including cell membrane debris, carbohydrates, small degraded nucleotides, host RNA, lipopolysaccharides, etc.

The expression “without effecting enzymatic digestion of RNA” refers to the absence of an enzyme such as RNase that digests RNA (including cellular or host RNA).

Performance is usually rated in terms of a nominal molecular weight cut-off (NMWC), which is defined as the smallest molecular weight species for which the filter membrane has more than 90% rejection.

A “purified” plasmid is one that is separated from contaminants such as endotoxin, protein, and RNA, and preferably composed of at least about 95% plasmid and about 5% RNA, more preferably at least about 98% plasmid and about 2% RNA, as measured by size-exclusion chromatography at 260 nm absorbance. Preferably, the endotoxin levels in such purified plasmid preparation are less than 300,000 EU/ml, more preferably less than 10,000 EU/ml.

A plasmid DNA can be highly purified in large yields from prokaryotic cells in which it is contained using TFF but without RNase. Preferably, the plasmid DNA herein has a size ranging from about 2 Kb to 50 Kb, more preferably about 2 to 15 Kb, and the TFF uses a selective nominal molecular weight cutoff of greater than about 100 k NMWC, preferably from about 300 k NMWC to 1000 k NMWC.

Plasmid DNA herein is isolated, or extracted, from components of prokaryotic cell cultures, preferably bacterial fermentations, and most preferably E. coli. Plasmid DNA isolated from prokaryotic cells includes naturally-occurring plasmids as well as recombinant plasmids containing a gene or genes of interest, including, e.g., marker genes or therapeutic genes. The fermentation may be carried out in any liquid medium that is suitable for growth of the cells being utilized.

The DNA plasmid to be purified herein may be any extrachromosomal DNA molecule of any character, provided that it is in the size range specified above. The plasmids may be high copy number, low copy number, and are double-stranded supercoiled plasmid DNA. They can contain a range of genetic elements that include selectable genes, polylinkers, origins of replication, promoters, enhancers, leader sequences, polyadenylation sites, and termination sequences. The plasmids can contain genes of basically any origin.

Before digestion and lysis of the cells to extract the plasmid DNA, the cells are generally first harvested from the fermentation medium. Any conventional means to harvest cells from a liquid medium is suitable, including centrifugation, filtration, and sedimentation.

The process herein involves lysis and solubilization of the cells, which results in chemical digestion of the RNA. This step is carried out for a time that ranges from about 30 seconds to 2 minutes, preferably from about 30 seconds to 1 minute. Typically, the cells are resuspended in buffer after harvest and treated for the indicated time period with one or more agents that function to lyse and solubilize the cells. Examples of such agents include alkali (e.g., dilute base such as sodium hydroxide) and/or a detergent. Preferably, both alkali and detergent are employed. In another preferred embodiment, for the maximum removal of endotoxin, the detergent is, for example, sodium dodecyl sulfate (SDS), cholic acid, deoxycholic acid, or TRITON X-114®, most preferably SDS or deoxycholic acid. For maximum plasmid release and removal of contaminating genomic DNA, the detergent is preferably anionic, more preferably SDS, cholic acid, or deoxycholic acid, and most preferably SDS or deoxycholic acid.

The lysing/solubilization is conducted in the absence of enzymes that digest RNA such as RNase. Preferably, the process is also carried out in the absence of enzymatic treatment that would weaken any cell wall which eliminates any possible animal viral contamination. It is also desirable to use methods that do not shear chromosomal DNA, so that its removal is facilitated and contamination with the final plasmid DNA product is avoided. The preferred lysis procedure for bacterial cells involves the alkaline lysis described in the following Examples.

After lysis, solubilization and neutralization, the cells are treated to remove lysate contaminants, including cellular debris such as proteins, cell walls, or membranes, chromosomal DNA, and host proteins. This removal step typically involves precipitation, centrifugation, filtration, and/or sedimentation depending on the cell type and the type of lysis employed. If alkali lysis is utilized, preferably the resultant lysate is mixed with an acidic solution to precipitate the chromosomal DNA and host proteins. Then cell debris and other impurities are preferably removed by standard means, such as centrifugation, filtration, or sedimentation, preferably centrifugation. The resultant supernatant is then optionally filtered with diatomaceous earth to clarify it and to reduce the concentration of host RNA with respect to the supernatant. The plasmid DNA can be precipitated from the clarified filtrate using a precipitating agent under suitable conditions, collected, and resuspended in a buffer. Subsequently, the host RNA, proteins, and lipopolysaccharides, as opposed to plasmid DNA, are preferably precipitated from the buffer with a precipitating agent under conditions appropriate for this purpose. Finally, the filtrate is collected for use in the TFF filtration step. The present invention is advantageous because it does not re-precipitate the plasmid DNA; therefore, the present invention is more cost effective.

The next step in the process involves filtering the solution through a TFF device. Prior to such filtering, the plasmid DNA may be treated with a short-chain polymeric alcohol, so that it does not bind to the TFF membrane as appropriate. The TFF process is described in detail in the following Examples. The filtration membrane is selected based on, e.g., the size and conformation of the plasmid DNA to be purified, and will have a molecular weight cut-off of greater than about 100 k NMWC, preferably about 300 k to 1000 k NMWC. They are typically synthetic membranes of either the microporous (MF) or the ultrafiltration (UF) type, with the reverse-osmosis (RO) type not normally applicable due to its small ranges of pore size.

An MF type has pore sizes typically from 0.1 to 10 micrometers, and can be made so that it retains all particles larger than the rated size. UF membranes have smaller pores and are characterized by the size of the globular protein that will be retained. They are available in increments from 1,000 to 1,000,000 nominal molecular weight (dalton) limits, corresponding approximately to 0.001 to 0.05 micrometers. UF membranes, which are normally asymmetrical with a thin film or skin on the upstream surface that is responsible for their separating power, are most commonly suitable for use in the present invention.

The process of the present invention is well adapted for use on a commercial or semi-commercial scale. It can be run continuously, i.e., on a continuous-flow basis of solution containing the desired plasmid DNA, past a tangential flow filter, until an entire, large batch has thus been filtered, followed by a stage of continuous flow separation of contaminants from desired plasmid DNA. Washing stages can be interposed between the filtration stages. Then fresh batches of solution can be treated. In this way, a continuous, cyclic process can be conducted, to give large yields of desired product, in acceptably pure form, in relatively short periods of time.

Under these conditions, plasmid DNA will be retained in the retentate while the contaminating substances, including many proteins, cell membrane debris, carbohydrates, small degraded nucleotides, etc., pass through the membrane into the filtrate. Commercial sources for filtration devices include Pall-Filtron (Northborough, Mass.), Millipore (Bedford, Mass.), and Amicon (Danvers, Mass.). Any filtration device useful for conducting TFF is suitable herein, including, e.g., a flat plate device, spiral wound cartridge, hollow fiber, tubular or single sheet device, open-channel device, etc.

The surface area of the filtration membrane used will depend on the amount of plasmid DNA to be purified. The membrane may be of a low-binding material to minimize adsorptive losses and is preferably durable, cleanable, and chemically compatible with the buffers to be used. A number of suitable membranes are commercially available, including, e.g., cellulose acetate, polysulfone, polyethersulfone, and polyvinylidene difluoride. Preferably, the membrane material is polysulfone or polyethersulfone.

Filtration is performed using tangential flow to circulate the sample buffer as it crosses the membrane surface. During TFF, pressure is applied across the membrane, which will allow smaller molecules to pass through the membrane while the retentate is recirculated. Typically, the flow rate will be adjusted to maintain a constant transmembrane pressure. Generally, filtration will proceed faster with higher pressures and higher flow rates, but higher flow rates are likely to cause shearing of the nucleic acid or loss due to passage through the membrane. In addition, various TFF devices may have certain pressure limitations on their operation. The pressure, therefore, may be adjusted according to the manufacturer's specification.

For flat-plate devices, the transmembrane pressure (TMP) is preferably about 5 to 30 psi, most preferably 10 to 15 psi. The circulation pump is selected to ensure minimal shearing of the nucleic acid. Typically, the circulation pump is a peristaltic pump or diaphragm pump in the feed channel and the pressure is controlled by adjusting the retentate valve.

Filtration will generally be performed in diafiltration mode. Optionally, the sample solution may initially be filtered without buffer addition until concentrated to a desired volume. Once concentrated, diafiltration buffer is added and filtration continues to wash the retentate of contaminating small molecules and remove unwanted solvents and salts. Diafiltration may be either continuous or discontinuous. Preferably, diafiltration is continuous, and performed until about 5 to 500 volume equivalents have been exchanged. Generally, more diafiltration will be performed with increased contaminants bound to the nucleic acids, depending on the purity required.

To further improve yield of the purified plasmid DNA following TFF, the retentate solution may optionally be recirculated through the filtration unit with the permeate valve closed for several minutes to remove residual plasmid DNA. The retentate is collected and additional diafiltration buffer is added to wash the membrane filter. The retentate is again collected and combined with the original retentate containing the purified plasmid DNA. The retentate may then be concentrated and then dialyzed against a buffer such as TRIS® to obtain purified plasmid DNA.

Plasmid DNA purified by the TFF process herein may be used directly or may be further purified depending on the level and type of contamination in the starting sample and the desired use. The plasmid DNA thus purified may be used for a number of applications, e.g., molecular biological applications such as cloning or gene expression, or for diagnostic applications using, e.g., PCR, RT-PCR, dendromer formation, etc. For therapeutic uses, e.g., for use in gene therapy or as a vaccine or in gene immunization, it may be desirable to further purify the plasmid DNA obtained from the TFF step. Reverse phase or ion-exchange chromatography may be used to further purify the plasmid DNA.

At a variety of places in the above protocol, analytical determination of plasmid DNA yield and purity are advantageously performed. Typically, such assays are performed before and after each purification step, as well as to each nucleic acid-containing fraction from, e.g., preparative ion-exchange chromatography. Preferred means for performing these analytical determinations include high-performance liquid chromatography (HPLC) or size-exclusion chromatography (SEC) analysis of purity, spectrophotometric estimation of yield, silver staining and SDS-PAGE for protein analysis, and agarose gel electrophoresis and Southern blotting for DNA analysis.

The invention will be more fully understood by reference to the following examples. They should not, however, be construed as limiting the scope of the invention. All literature and patent citations mentioned herein are expressly incorporated by reference.

Materials and Methods:

Bacterial Source:

A commercial E. coli strain harboring a 12.3 kb plasmid harvested from 5 L to 400 L fermenter was used for the development of the in-line lysis and neutralization processes. The plasmid DNA consists of pUC19 plasmid backbone with proviral DNA from a feline immunodeficiency virus.

Bacterial Harvest:

Bacterial cells used were harvested either by centrifugation or by filtration device with 0.1 μm to 300,000 NMWC filter.

Cell Suspension Solution:

Either frozen or fresh concentrated cells were suspended in cell suspension buffer prior to the lysis. Glucose-Tris-EDTA buffer (glucose 50 mM; Tris-HCl 25 mM; EDTA 10 mM) was used as cell suspension buffer. Eight ml of GTE per gram of bacterial cell paste was used for bacterial cell suspension.

Lysis Solution:

The lysis solution was prepared by addition of NaOH and SDS at a final concentration of 0.15N and 1% respectively. Sixteen ml of lysis solution was used per gram of cell pellets.

Neutralization Solution

Potassium acetate solution (3M, pH 5.5) was used as a neutralization solution at a concentration of 8 ml per gram of cell pellet.

Plasmid Isolation:

For the laboratory scale lysis and neutralization process, the plasmid in the neutralized lysate was isolated either by isopropanol precipitation or by Qiagen DNA isolation kit.

For the larger scale lysis-neutralization process, the plasmid was purified with TFF/Polyflo-column procedure.

Determination of Optimum Time for Lysis and Neutralization Time for Plasmid Isolation:

Experiment 1:

Objective: To determine the optimum lysis time for plasmid isolation.

Experimental Design:

TABLE 1

| Treatment | Lysis temperature | Lysis time |
|---|---|---|
| 1 | Ambient | 20, 40, 60, 120 |
| | temperature | and 360 minutes |
| 2 | 4° C. | 20, 40, 60, 120 |
| | | and 360 minutes |

Materials and Method:

One liter spinner flasks were used for lysis of bacterial cells. Twenty grams of E. coli cells were suspended in GTE buffer (8 ml per g) in a spinner flask either at room temperature or at 4 C. The cells were lysed by addition of lysis solution (16 ml per gram of cells). The samples were collected at 20 min, 40 min, 60 min, 120 min and 360 min. and neutralized by addition of neutralization solution. Plasmid DNA from aliquots was isolated and purified by isopropanol precipitation.

Results:

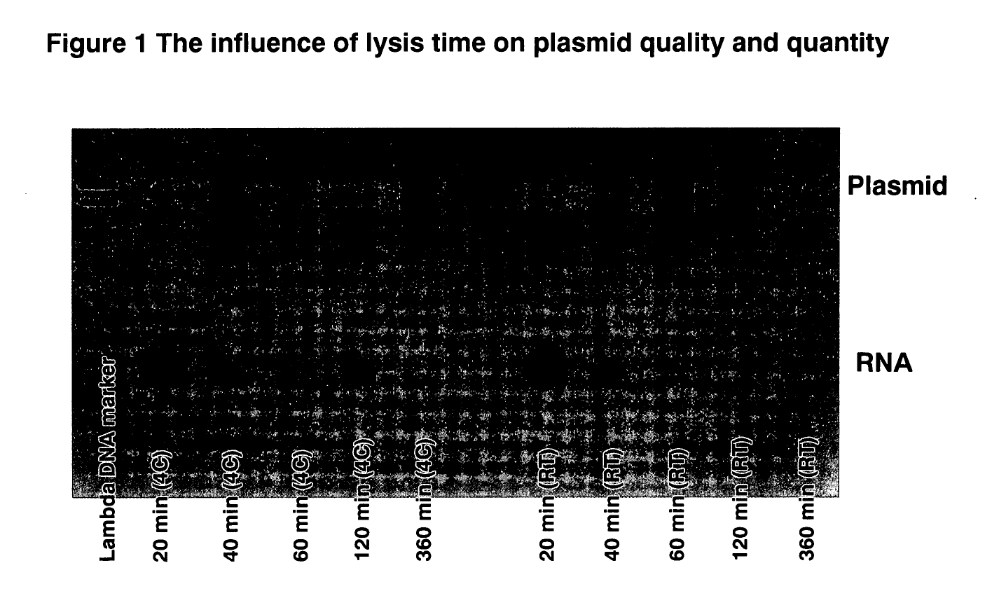

Results from lysis time experiment are depicted in FIG. 1. Lysis time of 20 min to 360 min had no effect on plasmid quality or quantity. The lysis time of 120 min or more at ambient temperature and 360 or more at 4° C. facilitated the degradation of the RNA in the neutralized cell lysate.

Experiment 2:

Objective: To determine the optimum neutralization time for plasmid isolation.

Experimental Design:

TABLE 2

| | Lysis/ | | |
|---|---|---|---|
| | neutralization | | Neutralization |
| Treatment | temperature | Lysis time | time |
| 1 | Ambient | 60 min | 20, 40, 60 and |
| | temperature | | 120 minutes |
| 2 | 4° C. | 60 min | 20, 40, 60 and |
| | | | 120 minutes |

Materials and Method:

One liter spinner flasks were used for lysis and neutralization of E. coli. Eighty grams of E. coli cells were suspended in GTE buffer (8 ml per g) in a spinner flask either at room temperature or at 4 C. The cells were lysed by addition of lysis solution (16 ml per gram of cells) for 60 min and neutralized. The samples were collected at 20 min, 40 min, 60 min, and 120 min. following neutralization. Plasmid DNA from aliquots was isolated and purified with a commercial Qiagen DNA isolation kit.

Results:

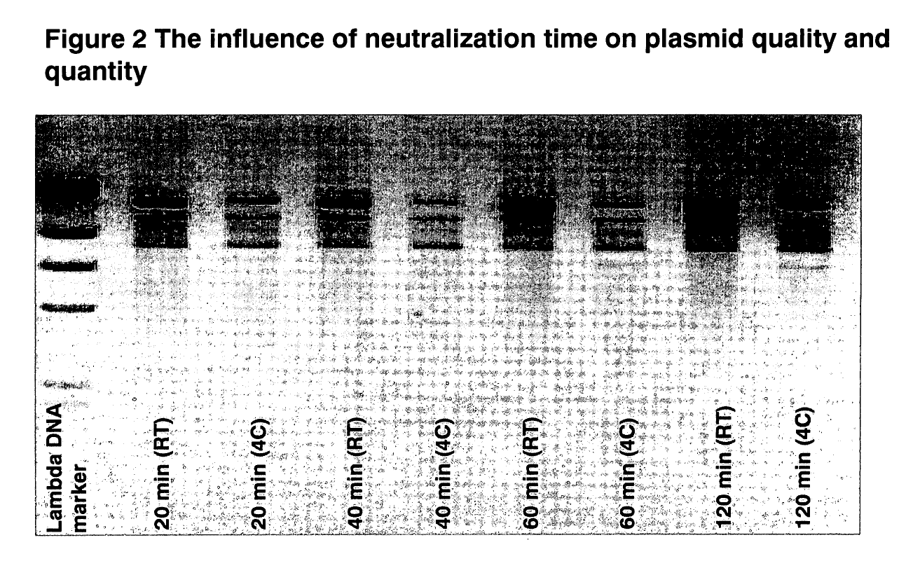

The results from neutralization time are depicted in FIG. 2. Neutralization time ranges from 20 to 120 minutes or neutralization temperature have no effect on the supercoiled isoform of plasmid. But there is an increase in the amount of non-supercoiled form plasmid DNA increases with increases in neutralization time at ambient temperature (RT).

Determination of Optimum Concentration of NaOH and Lysis Time for Plasmid Isolation:

Based on the results from experiments 1 and 2, experiment 3 was conducted to determine whether the lysis could be further reduced without affecting the quality of the plasmid DNA.

Experiment # 3

Objectives: 1) To determine optimum concentration of NaOH, lysis solution volume and lysis time on plasmid quality and yield.

Experimental Design:

TABLE 3

| | NaOH/SDS | Lysis | |
|---|---|---|---|
| | conc. in | solution | |
| | Lysis | volume per | |
| Treatment | solution | gram of paste | Lysis time |
| 1 | 0.2N/1% SDS | 16 ml | 1 min, 5 min, |
| | | | 10 min, 15 |
| | | | min, 30 min |
| 2 | 0.2N/1% SDS | 8 ml | 1 min, 5 min, |
| | | | 10 min, 15 |
| | | | min, 30 min |
| 3 | 0.15N/1% SDS | 16 ml | 1 min, 5 min, |
| | | | 10 min, 15 |
| | | | min, 30 min |
| 4 | 0.15N/1% SDS | 8 ml | 1 min, 5 min, |
| | | | 10 min, 15 |
| | | | min, 30 min |

Materials and Methods:

Bacterial cells were resuspended in GTE (8 ml per g paste) and lysed in a spinner flask as described in Table 3. Aliquots of samples were collected at 1 min, 5 min, 10 min, 15 min and 30 min. intervals and neutralized for plasmid isolation. The quality and quantity of the plasmid was visually analyzed following gel electrophoresis.

Results:

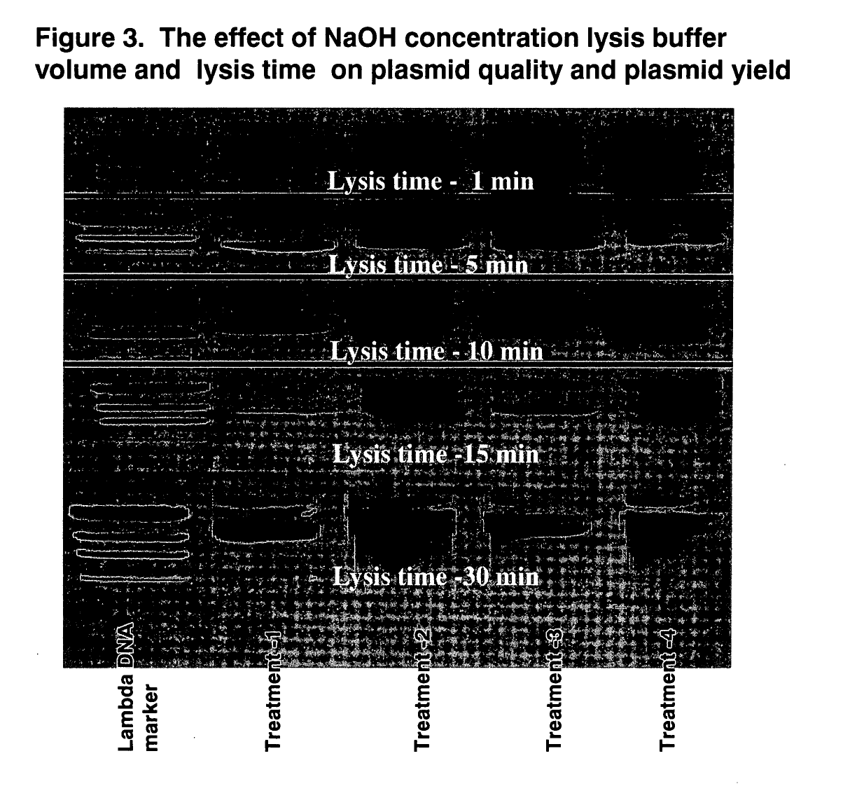

The quality of the plasmid resulted from experiment 3 is depicted in FIG. 3. Based on the gel electrophoresis results, it was concluded that a lysis time of 1 min should be sufficient for production of supercoiled isoform rich plasmid preparation. Sixteen ml of 0.15N NaOH/1% SDS lysis buffer per gram of cell was necessary for isolation of desirable quality plasmid. Also, for optimum quality and quantity of plasmid yield, 16 ml of lysis solution (0.15N NaOH/1% SDS) per gram of cell paste was necessary.

Laboratory Scale Lysis and Neutralization Process:

Based on the batch lysis and neutralization experiment, it was concluded that lysis duration of 1 min or less could be used for plasmid isolation without affecting the quality and plasmid yield. Thus, for plasmid isolation a continuous lysis and neutralization assemble could be used. To exemplify the use of a lysis and neutralization assembly, the following experiment was conducted.

Experiment 4:

Objective: To evaluate the lysis and neutralization process on plasmid quality and quantity.

Experimental Design:

TABLE 4

| | | Lysis | |
|---|---|---|---|
| | NaOH/SDS | solution | Neutralization |
| | conc. in | volume per | solution per |
| | Lysis | gram of cell | gram of cell |
| Treatment | solution | paste | paste |
| 1 | 0.2N/1% SDS | 16 ml | 8 ml |
| 2 | 0.2N/1% SDS | 8 ml | 8 ml |
| 3 | 0.15N/1% SDS | 16 ml | 8 ml |
| 4 | 0.15N/1% SDS | 8 ml | 8 ml |

Materials and Methods:

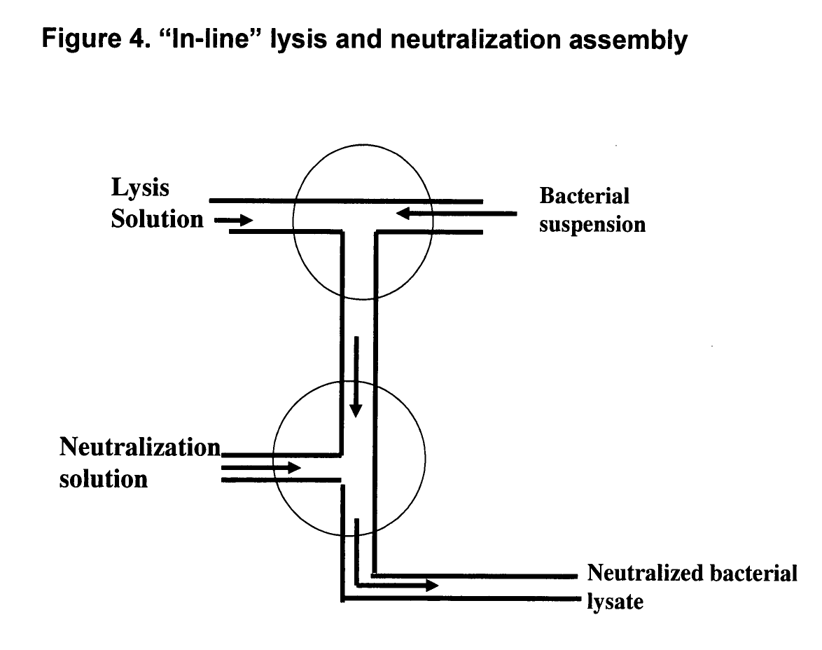

Polyethylene tubes with internal diameter ranges from 0.475 cm (0.25 inch) to 0.625 cm (0.188 inch) were used for the lysis and neutralization prototype. Lysis and neutralization prototype was assembled by connecting five polyethylene tubes ranging in size from 72 Cm to 90 cm and two “T” shaped connectors (FIG. 4). Three peristaltic pumps were used to pump the cell suspension, lysis solution and neutralization solution. The neutralized lysate was collected in 10 L carboys and aliquots of samples were analyzed for plasmid quality and quantity by gel electrophoresis.

Results:

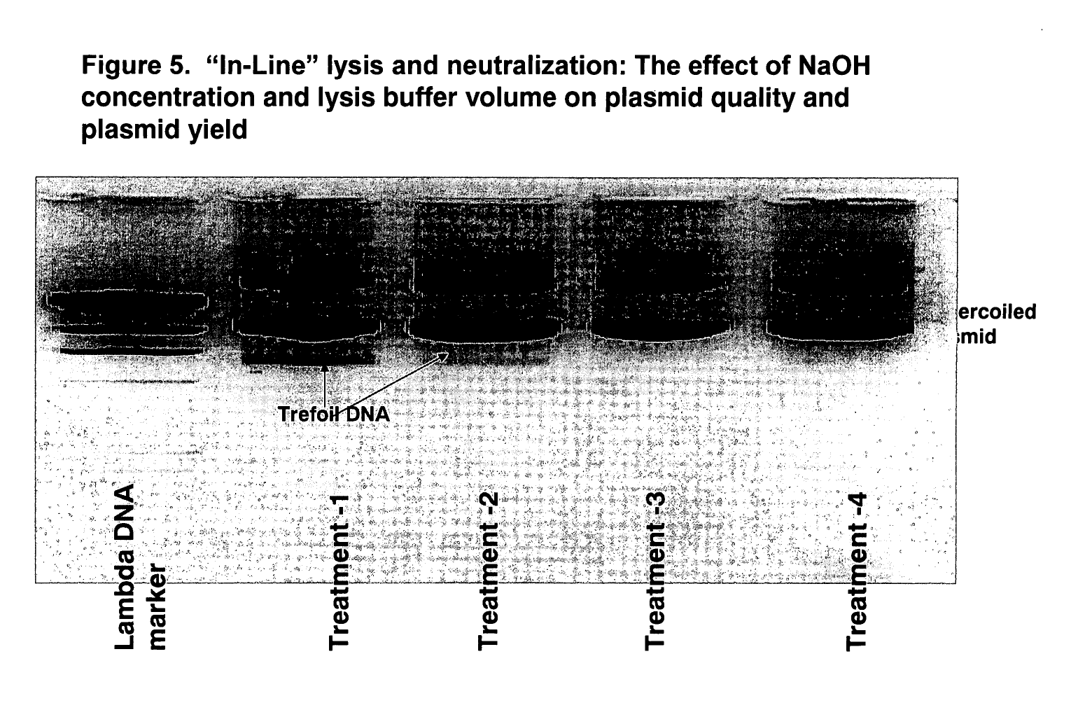

The plasmid isolated from Treatment-1 to -4 was depicted in FIG. 5. There was no noticeable difference in the quantity the plasmid due to various treatments. However, there was a noticeable difference in the quality of the plasmid isolated from Treatment 1 (0.2N NaOH and 1% SDS, 16 ml per gram of cell) and Treatment-2 (0.15N NaOH and 1% SDS, 8 ml per gram of cell). The amount of irreversibly denatured plasmid DNA (Trefoil DNA) was relatively in higher quantity in Treatment-1 compared to Treatment-2. The plasmid DNA isolated from Tretament-3 and -4 were free of “trefoil” DNA. It was concluded that lysis and neutralization prototype could be used for plasmid DNA isolation and 0.15N NaOH/1% SDS lysis solution at 16 ml per gram of cell was optimum for efficient lysis.

Experiment 5:

Objectives: The major objectives of this experiment were:
1) to evaluate the use of the lysis and neutralization process for isolation of plasmid from bacterial cells and
2) to determine the addition of motionless mixture(s) on the plasmid yield.

Experimental Design:

TABLE 5

| Treatment | Location of the motionless-mixture |
|---|---|
| 1 | N/A |
| 2 | Post-lysis |
| 3 | Post-neutralization |

Materials and methods: A polyethylene tube with internal diameter of 0.47 cm (0.188 inch) was used for lysis and neutralization steps. The prototype of the neutralization and lysis apparatus is depicted in FIG. 4. In-line lysis and neutralization prototype was assembled by connecting five polyethylene tubes ranging in size from 72 cm to 90 cm and two “T” shaped connectors. Three peristaltic pumps were used to pump the cell suspension (200 ml per minute), lysis solution (400 ml per minute) and neutralization solution (200 ml per minute). The neutralized lysate was collected in 20 L carboy and aliquots of samples were analyzed for plasmid quality.

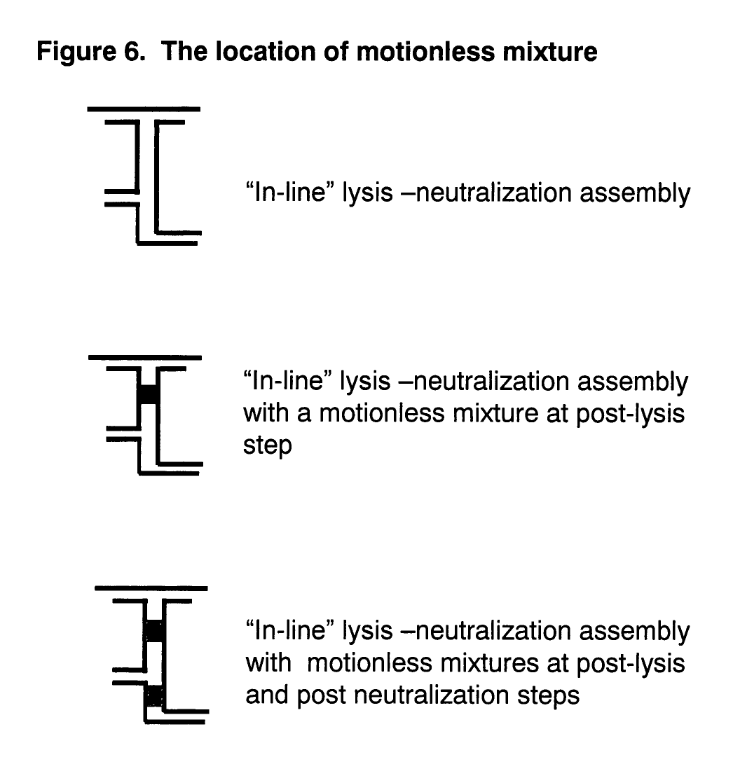

To determine the effect of motionless-mixtures on the plasmid yield, the motionless mixer was placed either at the tube carrying bacterial lysate or at the tube carrying neutralized lysate (FIG. 6).

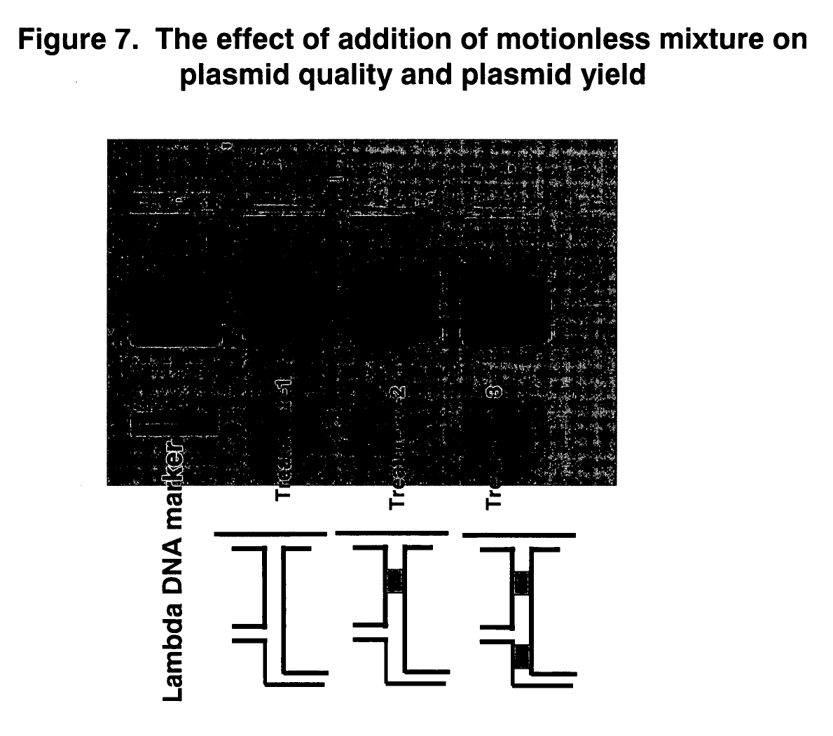

Gel electrophoresis: An aliquot of plasmid isolated from in-line lysis and neutralization process with or without motionless mixer was evaluated for the plasmid quality and quantity (FIG. 7).

Results: The visualization of plasmid DNA isolated from various treatments is depicted in FIG. 2. No difference in the quality of the DNA due to the treatments was detected. But the addition of motionless facilitated the thorough mixing (see FIG. 7) and resulted in higher plasmid yield (based on the band intensity).

Experiment 6:

Objectives: The major objectives of this experiment were: 1) to evaluate the use of “in-line” lysis and neutralization process for isolation of plasmid from bacterial cells compared to a batch method and 2) to determine the addition of motionless mixer(s) on the plasmid yield.

Experimental Design:

TABLE 6

| Treatment | Location of the motionless-mixer |
|---|---|
| 1 | N/A |
| 2 | Post-lysis |
| 3 | Post-lysis and post-neutralization |
| 4 | Post-neutralization |
| 5 | Batch mixing |

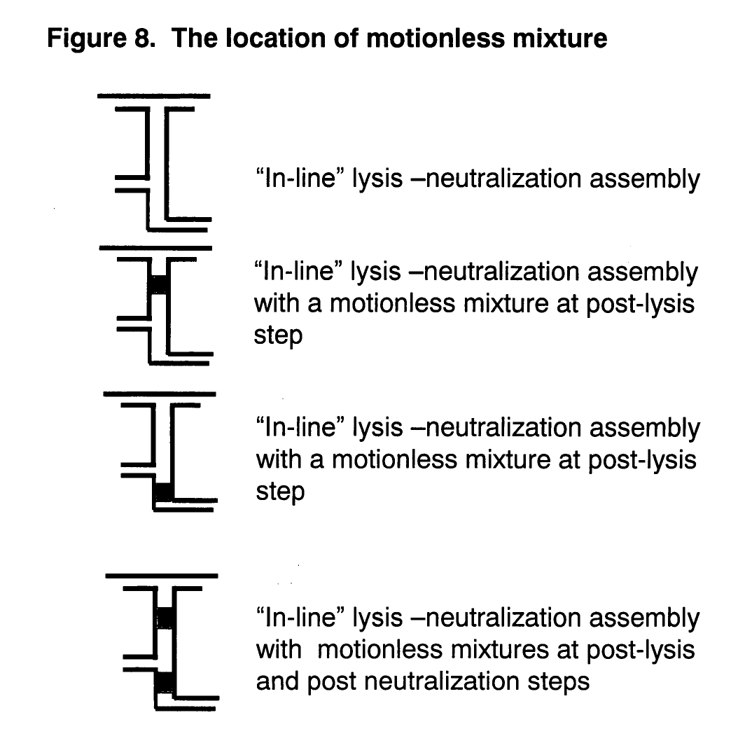

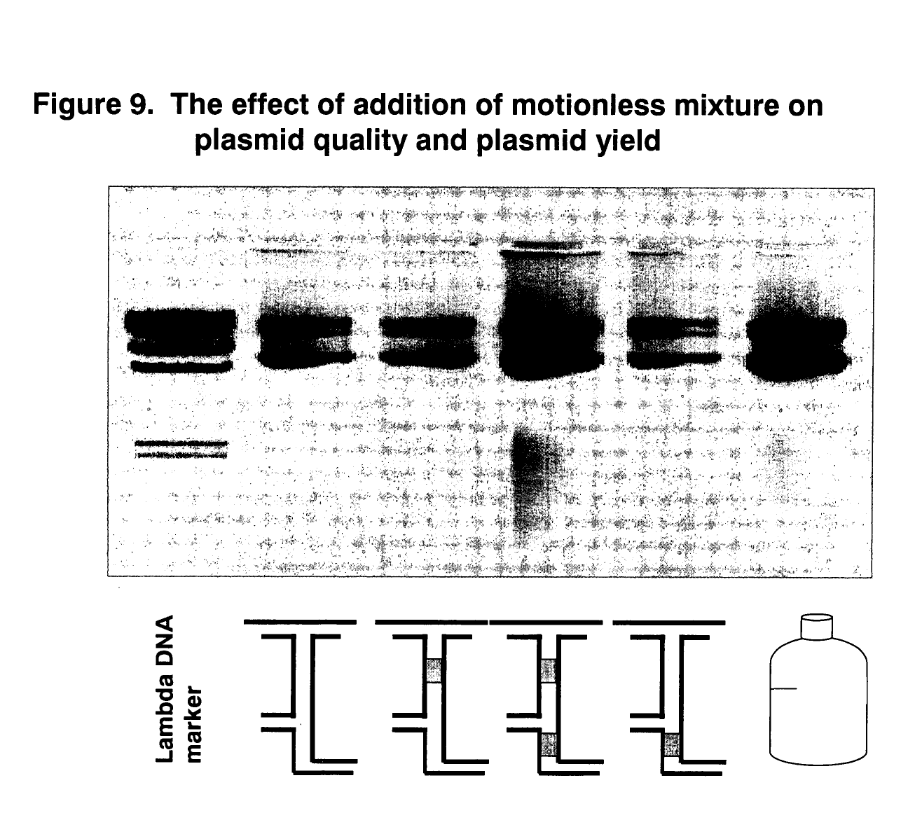

Materials and methods: Size and length of the polyethylene tubes, flow rate used in Experiment 6 were similar to that of Experiment 5. In addition, in Treatment 3, two motionless mixers were used and in Treatment 5, the lysis and neutralization procedure was carried out in 20 L carboys mimicking a batch process. For the batch process, the bacteria cell suspension and lysis solution were mixed in a 20 L carboy by inverting which was followed by addition of neutralization solution and through mixing by vigorous shaking to facilitate the formation of the protein-chromosomal DNA precipitate. The location of the motionless mixer in-line lysis and neutralization process was depicted in FIGS. 8 and 9.

Gel electrophoresis: An aliquot of plasmid isolated from in-line lysis and neutralization process with or without motionless mixer and batch process was evaluated for the plasmid quality and quantity (FIG. 5).

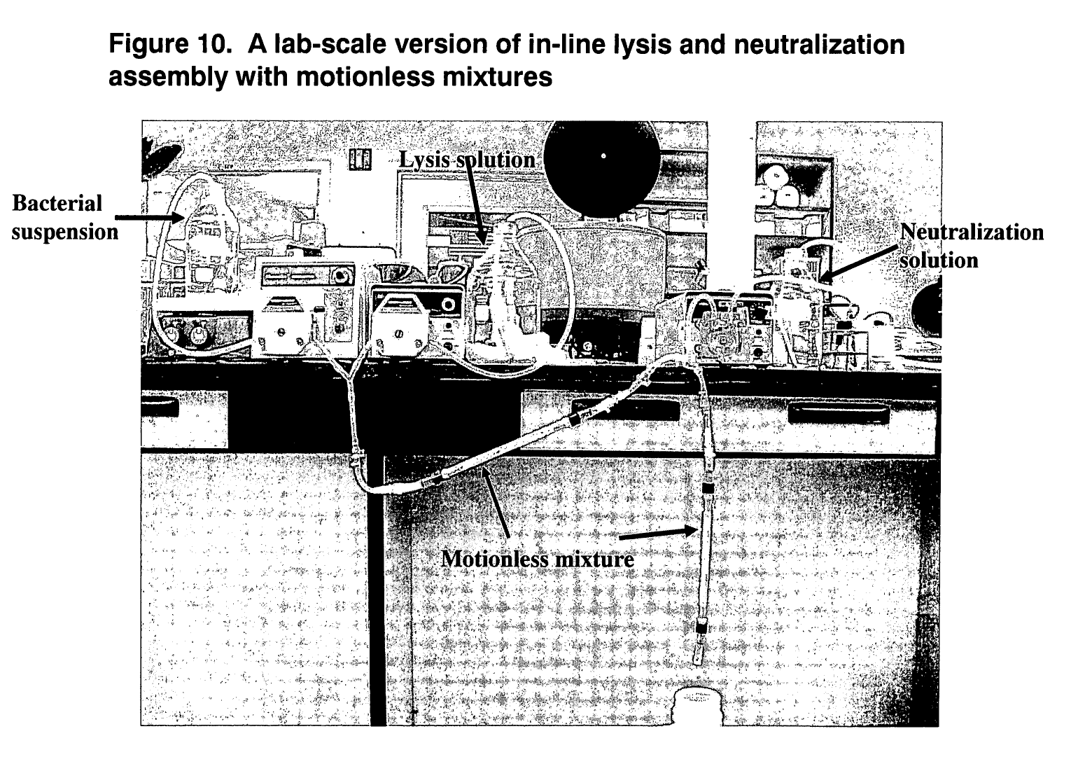

Results: The visualization of plasmid DNA isolated from various treatments is depicted in FIG. 9. There was no difference in the quality of the DNA due to the treatments. The plasmid yield was similar for batch process (Treatment 5) and the “in-line” lysis and neutralization process with two motionless mixers (Treatment 3). But the yield was relatively low for Treatments 1, 2 and 4. Addition of motionless mixers at post-lysis and post-neutralization improved the plasmid yield and was comparable to the treatment with no motionless mixers (Treatment 5). A lab scale “in-line” lysis and neutralization assembly is shown in FIG. 10.

### BIBLIOGRAPHY

Birnboim, H. C., and J. Doly, 1979. A rapid alkaline extraction procedure for screening recombinant plasmid DNA. Nucleic Acid Res. 7:1513-1523.
Bussey, L. B., R. Adamson, and A., Atchley, 2000. Methods for purifying nucleic acids. U.S. Pat. No. 6,011,148.
Butler, M. D., D. L. Cohen, D. Kahn, M. E. Winkler, 2001. Purification of Plasmid DNA. U.S. Pat. No. 6,313,285.
Ferreira, G. N. M., J. M. S. Cabral and D. M. F. Prazeres, 1999. Development of process flow sheets for the purification of supercoiled plasmids for gene therapy applications. Biotechnol. Prog 15:725-731.
Ferreira, G. N. M., G. A. Monteiro, D. M. F. Prazeres, and J. M. S. Cabral, 2000. Downstream processing of plasmid DNA for gene therapy and DNA vaccine applications. Trends Biotechnology 18:380-388.
Fieschko et al., 1986. Chem. Eng. Commun. 45:229-240
Hill A. F., M. Desbruslais, S. Joiner, K. C. Sidle, I. Gowland, J. Collinge, L. J. Doey and P. Lantos, 1997.
The same prion strain causes VCJD and BSE. Nature. 389:448-50.
Lee, A. L., and S. Sagar, 2001. Method for large scale plasmid purification. PCT 6,197,553
Levy, M S., R. D. O'Kennedy, Ayazi-Shamlou and P. Dunnill, 2000. Biochemical engineering approaches to the challenges of producing pure plasmid DNA. Trends Biotechnol. 18:296-305
Maniatis, T., E. F. Fritsch, And J. Sambrook, 1982. Molecular Cloning A Laboratory Manual. Cold Spring Harbor Laboratory Press, New York.
Morsey, M. A., 1999. Methods for production of recombinant plasmid. U.S. Pat. No. 5,922,583
Neidhardt, F. C., and H. E. Umbarger, 1996. Chemical composition of Escherichia coli. In Escherichia coli and Salmonella. Neidhardt, F. C. (Eds.). Volume 1, pages 13-16.
Riesenberg et al., 1991. Patent No. DD290215
Thatcher, D. R., A. Hitchcock, J. A. J. Hanak, and D. L. Varley, 1999. Method of plasmid DNA production and purification. U.S. Pat. No. 5,981,735
Thatcher, D. R., A. Hitchcock, J. A. J. Hanak, and D. L. Varley, 1999. Method of plasmid DNA production and purification. PCT WO 97/29190.
Theodossiou, O. R. T., and T. P. Dunnill, 1999. Methods of enhancing the recovery of plasmid genes from neutralized cell lysate. Bioprocess Engineering 20:147-156.
Wang B, Ugen, K E, Srikantan V, Agadjanyan M G, Dang K, Refaeli Y, Sato A I, Boyer J, Williams W V, Weiner D B 1993. Gene inoculation generates immune responses against human immunodeficiency virus type 1. Proc. Natl. Acad. Sci. USA. 90:4156-60.
Wei, C. 1999. Automated high-yield fermentation of plasmid DNA in Escherichia coli. U.S. Pat. No. 5,955,32.

1. A process for purifying plasmid DNA from prokaryotic cells, comprising:
(a) digesting the cells;
(b) incubating the cells in the presence of alkali and a detergent for about 30 seconds to 2 minutes to effect lysis and solubilization thereof;
(c) removing lysate contaminants from the cells to provide a plasmid DNA solution;
(d) filtering the solution through a tangential flow filtration device to obtain a retentate containing the plasmid DNA; and
(e) collecting the retentate, whereby enzymes are not used in any of the above steps to digest RNA.

2. The process of claim 1, wherein the cells are bacterial cells.

3. The process of claim 1, wherein the cells are E. coli cells.

4. The process of claim 1, wherein the plasmid DNA has a size ranging from about 2 to 15 kilobases.

5. The process of claim 1, wherein step (b) is carried out for about 30 seconds to 1 minute.

6. The process of claim 1, wherein the process is a continuous process.

7. The process of claim 1, wherein the process is a batch process.

8. The process of claim 1, wherein the filtration device has a membrane with a nominal molecular weight cutoff of greater than about 100 k NMWC.

10. The process of claim 1, further comprising recovering the plasmid DNA from the retentate.

11. The process of claim 1, further comprising subjecting the retentate to reverse-phase chromatography.

12. The process of claim 1, wherein the detergent is ionic.

13. The process of claim 1, wherein the detergent is anionic.

14. The process of claim 14, wherein the detergent is sodium dodecyl sulfate, cholic acid, or deoxycholic acid.

15. The process of claim 1, wherein the plasmid DNA has a molecular weight over 7000 kDa.
